(19) United States
(12) Patent Application Publication
(10) Pub. No.: US 2026/0325036 A1
(43) Pub. Date: Oct. 22, 2026

(54) SYSTEM AND METHOD FOR DETERMINING REAL-TIME ELECTROCARDIOGRAM (ECG) DATA USING MULTIPLE ANATOMICAL CONTACT ELECTRODES

(71) Applicant: Aarogya Tech Inc., Reading, PA (US)

(72) Inventors: Amit Kumar Gupta, Bangalore (IN); Prashant Trivedi, Reading, PA (US)

(21) Appl. No.: 19/529,203

(22) Filed: Feb. 4, 2026

Related U.S. Application Data
(60) Provisional application No. 63/753,978, filed on Feb. 5, 2025.

(51) Int. Cl. A61B 5/318; A61B 5/00; A61B 5/259; A61B 5/282; A61B 5/308; A61B 5/339; G16H 40/67

(52) U.S. Cl. CPC A61B 5/318; A61B 5/0006; A61B 5/259; A61B 5/282; A61B 5/308; A61B 5/339; A61B 5/6828; A61B 5/6829; A61B 5/6843; A61B 5/746; G16H 40/67; A61B 5/6891

(57) ABSTRACT

A system and a method for determining real-time electrocardiogram (ECG) data using multi-contact electrodes are disclosed. The system comprises a pair of first electrodes integrated into opposing armrests of a user support device, configured to provide electrical contact with palms or wrists for capturing cardiac biopotential voltage signals. A pair of second electrodes is integrated into a calf support, configured to provide electrical contact with calves or ankles for capturing cardiac biopotential voltage signals. An electrocardiogram module is electrically coupled to both electrode pairs, configured to simultaneously condition the cardiac biopotential voltage signals. An analog-to-digital converter is connected to the electrocardiogram module, configured to digitize the conditioned signals. A control unit is connected to the analog-to-digital converter, configured to digitally process the digitized signals to detect cardiac peaks or reconstruct a real-time ECG waveform. The integrated seating configuration enables repeated or continuous ECG measurements in relaxed environments.

### CROSS REFERENCE TO RELATED APPLICATION(S)

This application claims the priority to and incorporates by reference the entire disclosure of U.S. provisional patent application bearing No. 63/753,978 filed on February 05, 2025.

### TECHNICAL FIELD

Embodiments of the present disclosure relate to Electrocardiogram (ECG) systems, and more particularly relate to a system and a method for determining real-time ECG data using multi-contact electrodes integrated into a user support device.

### BACKGROUND

Electrocardiogram (ECG) measurement is a widely used technique for monitoring cardiac activity and diagnosing cardiovascular conditions. Conventional ECG systems typically employ multiple electrodes affixed to the skin of a user at predefined anatomical locations using adhesive patches, straps, or conductive gels. Such electrodes are commonly positioned on the chest, arms, and lower limbs to acquire cardiac biopotential signals.

Although conventional ECG systems provide clinically reliable measurements, they present several technical and practical limitations. In particular, the attachment of adhesive electrodes requires careful skin preparation and precise placement, which increases setup time and limits usability for non-professional users. Prolonged use of adhesive materials may also cause skin irritation, discomfort, and reduced signal quality due to sweat, movement, or loosening of the electrodes.

Furthermore, many existing ECG systems require users to maintain a fixed posture during measurement in order to minimize motion artifacts and signal disturbances. Such posture requirements can be difficult to satisfy for elderly users, individuals with mobility limitations, or users undergoing long-duration monitoring. Variations in posture and involuntary movements frequently result in unstable electrode contact and degraded signal quality.

Various portable and wearable ECG devices have been proposed to improve accessibility outside clinical environments. However, such devices often employ limited electrode configurations or reduced sensing areas, which may compromise signal accuracy and reliability. In addition, wearable devices are susceptible to displacement during daily activities and may require frequent adjustment or recalibration.

Some prior art systems disclose ECG monitoring arrangements integrated into seating structures or furniture components, such as armrests or seat cushions. For example, a chair-based electrocardiogram system incorporating electrodes in seat portions for acquiring cardiac signals. While such approaches reduce the need for adhesive electrodes, they generally rely on limited contact locations and lack mechanisms for maintaining consistent signal quality during prolonged use.

Moreover, existing seating-based ECG systems typically provide minimal feedback regarding electrode contact quality and user positioning. As a result, users may remain unaware of suboptimal contact conditions, leading to inaccurate measurements and repeated recording sessions. The absence of automated contact verification and adaptive signal quality management further limits the effectiveness of such systems in non-clinical environments.

In addition, conventional ECG technologies are often designed either for short-duration diagnostic sessions in medical facilities or for intermittent monitoring using portable devices. These systems are not optimized for continuous or repeated measurements in relaxed, seated environments such as homes, offices, or assisted-living facilities. Consequently, opportunities for routine and preventive cardiac monitoring remain underutilized.

Accordingly, there exists a need for an ECG monitoring solution that improves ease of use, reduces setup complexity, and enhances signal reliability in seated environments. Such a solution should facilitate consistent acquisition of high-quality cardiac data without requiring specialized training, extensive preparation, or uncomfortable electrode attachment procedures.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a system and a method for determining real-time ECG data using multi-contact electrodes integrated into a user support device are disclosed.

In accordance with an embodiment of the present disclosure, the system comprises a pair of first electrodes, a pair of second electrodes, an electrocardiogram (ECG) module, an analog-to-digital converter, and a control unit.

In one aspect, the pair of first electrodes of the multiple anatomical contact electrodes is integrated into opposing armrests of a user support device. The pair of first electrodes is configured to provide an electrical contact with one of: palms and wrists of a user for capturing cardiac biopotential voltage signals. Each first electrode of the pair of first electrodes comprises an electrically conductive material constructed from one of: copper, silver, silver chloride, stainless steel, silver-coated fibers, conductive polymers, and carbon. The pair of first electrodes is positioned on upper surfaces of the opposing armrests at predetermined locations and orientations corresponding to average palm and wrist resting positions of the user, the pair of first electrodes configured to establish the electrical contact in response to the user resting arms on the opposing armrests.

In another aspect, the pair of second electrodes of the multiple anatomical contact electrodes is integrated into a calf support of the user support device. The pair of second electrodes is configured to provide the electrical contact with one of: calves and ankles of the user for capturing the cardiac biopotential voltage signals. Each second electrode of the pair of second electrodes comprises a semicircular arch configured with a conductive tape constructed from one of: the copper, the silver, the silver chloride, the stainless steel, the silver-coated fibers, the conductive polymers, and the carbon. The pair of second electrodes further comprises a resilient biasing member configured to urge the conductive tape toward a surface of the calf support to provide a user-perceptible tactile response upon establishing the electrical contact. The calf support is dimensioned and contoured according to anthropometric reference data to accommodate placement of one of: the calves and the ankles of the user for maintaining consistent skin-electrode impedance during use.

Yet another aspect, the ECG module is electrically coupled to the pair of first electrodes and the pair of second electrodes. The ECG module is configured to simultaneously condition the cardiac biopotential voltage signals received from the pair of first electrodes and the pair of second electrodes. The ECG module comprises an instrumentation amplifier and at least one analog. The instrumentation amplifier is configured to differentially amplify the cardiac biopotential voltage signals. The at least one analog filter is configured to at least one of: attenuate baseline drift, power-line interference, and elevated-frequency noise.

Yet another aspect, the analog-to-digital converter is operatively connected to the electrocardiogram (ECG) module, and configured to digitize the conditioned cardiac biopotential voltage signals.

Yet another aspect, the control unit is operatively connected to the analog-to-digital converter. The control unit is configured to digitally process the digitized cardiac biopotential voltage signals to at least one of: detect cardiac peaks and reconstruct a real-time ECG waveform for determining real-time ECG data using the multiple anatomical contact electrodes. The control unit includes one or more adaptive contact-detection models configured to monitor at least one of: the skin-electrode impedance, signal-to-noise ratio, and the baseline drift at defined electrode contact locations on the user, identify suboptimal electrical contact, and to generate a corrective output as one or more alerts in response thereto. The control unit is configured to transmit the one or more alerts prompting the user to adjust one of: a posture and a contact of at least one of: the palms, the wrists, the calves, and the ankles with the pair of first electrodes and the pair of second electrodes to maintain optimal signal quality. The one or more alerts comprise at least one of: one or more visual alerts and one or more audio alerts transmitted in real-time. The control unit is configured to transmit the real-time ECG data to a cloud server for at least one of: storage, processing, analysis, and medical records Yet another aspect, the system further comprises a display unit operatively attached to the user support device, configured to display the real-time ECG waveform and the one or more alerts.

In accordance with another embodiment of the present disclosure, the method for determining the real-time ECG data using the multiple anatomical contact electrodes is disclosed. In the first step, the method includes providing, by the pair of first electrodes of the multiple anatomical contact electrodes integrated into opposing armrests of the user support device, the electrical contact with one of: the palms and the wrists of the user to capture cardiac biopotential voltage signals.

In the next step, the method includes providing, by the pair of second electrodes of the multiple anatomical contact electrodes integrated into the calf support of the user support device, the electrical contact with one of: the calves and the ankles of the user to capture the cardiac biopotential voltage signals.

In the next step, the method includes simultaneously conditioning, by the ECG module, the cardiac biopotential voltage signals received from the pair of first electrodes and the pair of second electrodes.

In the next step, the method includes digitizing, by the analog-to-digital converter operatively connected to the ECG module, the conditioned cardiac biopotential voltage signals.

In the next step, the method includes digitally processing, by the control unit operatively connected to the analog-to-digital converter, the digitized cardiac biopotential voltage signals to at least one of: detect cardiac peaks and reconstruct the real-time ECG waveform to determine the real-time ECG data using the multiple anatomical contact electrodes.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 illustrates an exemplary block diagram depicting a system for determining real-time electrocardiogram (ECG) data using multiple anatomical contact electrodes, in accordance with an embodiment of the present disclosure;

FIG. 2A illustrates an exemplary user support device with the multiple anatomical contact electrodes, in accordance with an embodiment of the present disclosure;

FIGS. 2B to 2D illustrate exemplary visual representations depicting opposing armrests of the user support device integrated with a pair of first electrodes, in accordance with an embodiment of the present disclosure;

FIG. 2E illustrates an exemplary visual representation depicting a calf support along with a pair of second electrodes attached to the user support device, in accordance with an embodiment of the present disclosure;

FIG. 2F illustrates an exemplary visual representation depicting a display unit attached to the user support device, in accordance with an embodiment of the present disclosure; and

FIG. 3 illustrates an exemplary flowchart of a method for determining the real-time ECG data using the multiple anatomical contact electrodes, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises… a" does not, without more constraints, preclude the existence of other devices, sub-systems, additional sub-modules. Appearances of the phrase "in an embodiment”, "in another embodiment" and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

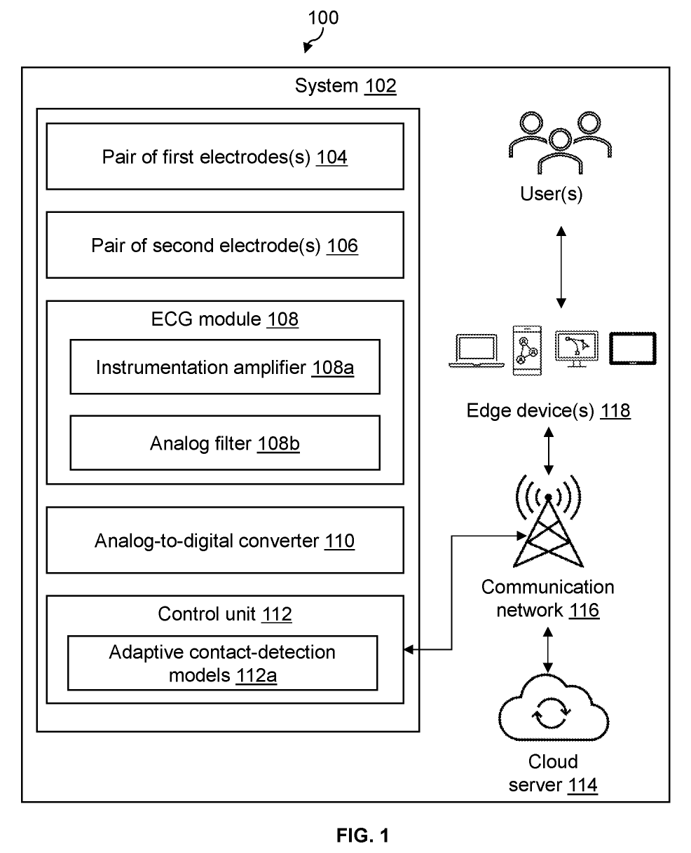

Referring now to the drawings, and more particularly to FIG. 1 through FIG. 3, where similar reference characters denote corresponding features consistently throughout the figures, there are shown preferred embodiments, and these embodiments are described in the context of the following exemplary system and/or method.

FIG. 1 illustrates an exemplary block diagram 100 depicting a system 102 for determining real-time electrocardiogram (ECG) data using multiple anatomical contact electrodes (104 and 106), in accordance with an embodiment of the present disclosure.

According to an exemplary embodiment of the present disclosure, the system 102 comprises a pair of first electrodes 104, a pair of second electrodes 106, an ECG module 108, an analog-to-digital converter 110, and a control unit 112. The system 102 may further include a cloud server 114, a communication network 116, and one or more edge devices 118 for data transmission, storage, and user access.

In an exemplary embodiment, the pair of first electrodes 104 of the multiple anatomical contact electrodes (104 and 106) may be integrated into opposing armrests of a user support device. The pair of first electrodes 104 may be configured to provide an electrical contact with one of: palms and wrists of a user for capturing cardiac biopotential voltage signals. The pair of second electrodes 106 of the multiple anatomical contact electrodes (104 and 106) may be integrated into a calf support of the user support device. The pair of second electrodes 106 may be configured to provide the electrical contact with one of: calves and ankles of the user for capturing the cardiac biopotential voltage signals.

In an illustrative embodiment, the multiple anatomical contact electrodes (104 and 106) comprise four distinct electrodes configured for electrocardiogram signal acquisition. A left arm electrode of the pair of first electrodes 104 may be mounted on a left armrest of the user support device 208 and configured to establish electrical contact with one of a palm and fingers of a left arm of the user. A right arm electrode of the pair of first electrodes 104 may be mounted on a right armrest of the user support device 208 and configured to establish electrical contact with one of a palm and fingers of a right arm of the user. A left leg electrode of the pair of second electrodes 106 may be mounted on the user support device and configured to establish electrical contact with a left leg calf of the user. A right leg electrode of the pair of second electrodes 106 may be mounted on the user support device 208 and configured to establish electrical contact with one of a right arm forearm and a right leg calf of the user. In some exemplary aspects, the right leg electrode may be positioned on the right armrest to enable forearm contact in addition to or as an alternative to right leg calf contact.

In an exemplary embodiment, the left arm electrode, the right arm electrode, and the left leg electrode may function as measurement electrodes configured to capture the cardiac biopotential voltage signals from respective anatomical contact locations. The right leg electrode may function as a driver electrode configured to provide a reference potential for improving noise rejection and signal quality during electrocardiogram signal acquisition. The driver electrode may inject a common-mode rejection signal to reduce interference from external electromagnetic sources and power-line noise. The configuration of three measurement electrodes and one driver electrode may enable acquisition of multiple electrocardiogram lead signals while maintaining signal integrity through active noise cancellation.

In an exemplary embodiment, the pair of first electrodes 104 and the pair of second electrodes 106 may be operatively connected to the ECG module 108 via a hardwired connection. The ECG module 108 may be electrically coupled to the pair of first electrodes 104 and the pair of second electrodes 106. The ECG module 108 may be configured to simultaneously condition the cardiac biopotential voltage signals received from the pair of first electrodes 104 and the pair of second electrodes 106.

In an exemplary embodiment, the ECG module 108 may comprise an instrumentation amplifier 108a and at least one analog filter 108b. The instrumentation amplifier 108a may be configured to differentially amplify the cardiac biopotential voltage signals received from the pair of first electrodes 104 and the pair of second electrodes 106. Differential amplification by the instrumentation amplifier 108a may enhance the cardiac biopotential voltage signals while rejecting common-mode noise present at electrode contact locations.

In an exemplary embodiment, the at least one analog filter 108b may be configured to attenuate at least one of baseline drift, power-line interference, and elevated-frequency noise. The at least one analog filter 108b may include one or more filter stages configured to remove unwanted signal components from the cardiac biopotential voltage signals. In some cases, the at least one analog filter 108b may include a high-pass filter component configured to attenuate baseline drift caused by electrode motion or respiration. In some cases, the at least one analog filter 108b may include a notch filter component configured to attenuate power-line interference at frequencies such as 50 Hz or 60 Hz. In some cases, the at least one analog filter 108b may include a low-pass filter component configured to attenuate elevated-frequency noise from muscle activity or electromagnetic interference.

In an exemplary embodiment, the ECG module 108 may continuously collect the cardiac biopotential voltage signals from the pair of first electrodes 104 and the pair of second electrodes 106. The ECG module 108 may perform signal conditioning to reduce noise and amplify relevant cardiac biopotential voltage signals for accurate monitoring. The signal conditioning performed by the ECG module 108 may include amplification by the instrumentation amplifier 108a followed by filtering by the at least one analog filter 108b.

The ECG module 108 may analyze the conditioned cardiac biopotential voltage signals to create the real-time ECG waveform. The conditioned cardiac biopotential voltage signals may be transmitted from the ECG module 108 to the analog-to-digital converter 110 for digitization. The control unit 112 may then process the digitized cardiac biopotential voltage signals to reconstruct the real-time ECG waveform for display and analysis.

In an exemplary embodiment, the analog-to-digital converter 110 may be operatively connected to the ECG module 108. The analog-to-digital converter 110 may be configured to digitize the conditioned cardiac biopotential voltage signals received from the ECG module 108. The ECG module 108 conditions the cardiac biopotential voltage signals by performing differential amplification and analog filtering to produce stabilized analog signals within a predefined voltage range suitable for digital conversion. The analog-to-digital converter 110 receives the conditioned cardiac biopotential voltage signals and samples the signals at a predetermined sampling frequency selected to preserve waveform morphology and temporal characteristics of the electrocardiogram signal.

In an exemplary embodiment, the control unit 112 may be operatively connected to the analog-to-digital converter 110. The control unit 112 may be configured to digitally process the digitized cardiac biopotential voltage signals to at least one of detect cardiac peaks and reconstruct a real-time ECG waveform for determining real-time ECG data using the multiple anatomical contact electrodes (104 and 106). The control unit 112 may include, but not limited to, one of: a microprocessor, a microcomputer, a microcontroller, a digital signal processor, a central processing unit, a state machine, a logic circuit, and the like.

In an illustrative embodiment, the system 102 may be configured to measure a potential difference between the right arm electrode and the left arm electrode, referred to as Lead I. The Lead I may represent a horizontal electrical vector across the heart and may be utilized for detecting cardiac rhythm and identifying certain arrhythmia conditions. The system 102 may further be configured to measure a potential difference between the right arm electrode and the left leg electrode, referred to as Lead II. The Lead II may represent a diagonal electrical vector from the right arm to the left leg and may provide enhanced visibility of P-waves and ventricular depolarization (QRS complexes) in the ECG waveform. The control unit 112 may process the digitized cardiac biopotential voltage signals to compute the Lead I and the Lead II waveforms based on differential voltage measurements between the pair of first electrodes 104 and the pair of second electrodes 106.

In an exemplary embodiment, the control unit 112 may be configured to transmit the real-time ECG data to the cloud server 114 for at least one of: storage, processing, analysis, and medical records. The control unit 112 may transmit the real-time ECG data to the cloud server 114 via a wireless connectivity module. The communication network 116 may facilitate data transmission between the control unit 112 and the cloud server 114. The system 102 may use edge-based or cloud-based signal processing for analyzing the real-time ECG data.

In an exemplary embodiment, the control unit 112 may communicate the real-time ECG data to the display unit via one or more data interfaces. The one or more data interfaces may include, but not limited to, Recommended Standard 232 (RS232), universal serial bus (USB), and similar wired communication protocols. The RS232 interface may provide serial data transmission between the control unit 112 and the HMI unit for displaying the real-time ECG waveform, heart rate, respiration rate, and system status information. The USB interface may provide higher-bandwidth data transmission and may enable connection to external computing devices for data logging, analysis, or firmware updates. In some exemplary aspects, the control unit 112 may support multiple simultaneous data interfaces to enable communication with both a local display units and the one or more end devices 118 via the communication network 116.

In an exemplary embodiment, the one or more edge devices 118 may be communicatively coupled to the communication network 116. The one or more edge devices 118 may provide an interface for one or more users to access the real-time ECG data transmitted by the control unit 112.

In an exemplary embodiment, the control unit 112 includes one or more adaptive contact-detection models 112a implemented as executable software modules, firmware routines, or hardware-assisted processing circuits configured to evaluate electrical contact quality between the user and the pair of first electrodes 104 and the pair of second electrodes 106. The adaptive contact-detection models 112a are configured to continuously receive digitized cardiac biopotential voltage signals and associated measurement parameters from the ECG module 108 and the analog-to-digital converter 110. Based on the received signals, the adaptive contact-detection models 112a monitor at least one of skin-electrode impedance, signal-to-noise ratio, and baseline drift at defined electrode contact locations on the user.

In an exemplary embodiment, the skin-electrode impedance refers to an electrical resistance and reactance present at an interface between an electrode surface and a skin surface of the user, and is determined by injecting a low-amplitude test current through the electrodes and measuring a resulting voltage response. The signal-to-noise ratio refers to a ratio between an amplitude of cardiac-related signal components and an amplitude of non-cardiac noise components within the digitized cardiac biopotential voltage signals. The baseline drift refers to low-frequency variations in a baseline level of the electrocardiogram waveform caused by electrode motion, respiration, or changes in contact pressure. The defined electrode contact locations correspond to physical interfaces between the pair of first electrodes 104 and palms or wrists of the user and between the pair of second electrodes 106 and calves or ankles of the user.

In an exemplary embodiment, the adaptive contact-detection models 112a analyze the monitored at least one of skin-electrode impedance, signal-to-noise ratio, and baseline drift by applying predefined mathematical procedures and threshold comparison procedures to identify suboptimal electrical contact. The adaptive contact-detection models 112a compare measured skin-electrode impedance values with predetermined impedance threshold values stored in a memory associated with the control unit 112. When the measured impedance exceeds the predetermined threshold value, the adaptive contact-detection models 112a determine that electrical coupling between the electrode and the skin is insufficient.

In an exemplary embodiment, the adaptive contact-detection models 112a determine signal-to-noise ratio by calculating a ratio between spectral energy associated with cardiac frequency bands and spectral energy associated with noise frequency bands using frequency-domain or time-domain analysis techniques. When the calculated signal-to-noise ratio falls below a predetermined threshold value, the adaptive contact-detection models 112a identify degradation in signal quality. The baseline drift is determined by measuring slow-varying changes in waveform offset using moving-average filtering or polynomial fitting techniques. When baseline drift exceeds acceptable limits stored in the control unit 112, the adaptive contact-detection models 112a identify instability in electrode contact.

In an exemplary embodiment, the adaptive contact-detection models 112a adaptively update the predetermined threshold values based on historical measurement data, user-specific physiological characteristics, and environmental conditions. For example, threshold values may be automatically adjusted in response to repeated measurements indicating stable contact at higher impedance levels for a particular user. This adaptive adjustment enables personalized contact quality assessment.

For example, during an ECG measurement session, the adaptive contact-detection models 112a may determine that skin-electrode impedance at one of the second electrodes 106 exceeds approximately 2 megaohms, that signal-to-noise ratio falls below approximately 10 decibels, and that baseline drift exceeds approximately 0.5 millivolts. Based on these measured values exceeding respective predetermined thresholds, the adaptive contact-detection models 112a classify the electrical contact as suboptimal and generate a corrective output.

The adaptive contact-detection models 112a further include classification logic configured to correlate multiple monitored parameters to distinguish between transient disturbances and persistent contact deficiencies. For example, short-duration impedance fluctuations caused by minor user movement may be ignored, while sustained deviations exceeding a predefined duration threshold are classified as contact failure events. This classification logic enables reliable detection of meaningful contact degradation while minimizing false alerts.

The configuration of the adaptive contact-detection models 112a as described herein enables continuous, automated evaluation of electrode contact quality at multiple anatomical locations, thereby ensuring stable acquisition of cardiac biopotential voltage signals and reliable determination of real-time electrocardiogram data.

In an exemplary embodiment, the control unit 112 may be configured to generate a corrective output as one or more alerts in response to the adaptive contact-detection models 112a identifying suboptimal electrical contact. The one or more alerts may comprise at least one of one or more visual alerts and one or more audio alerts transmitted in real-time. The one or more visual alerts may be displayed on a display unit operatively attached to the user support device or on a user interface associated with the one or more edge devices 118. The one or more audio alerts may be emitted by a speaker associated with the user support device or the one or more edge devices 118.

The control unit 112 may be configured to transmit the one or more alerts prompting the user to adjust one of a posture and a contact of at least one of the palms, the wrists, the calves, and the ankles with the pair of first electrodes 104 and the pair of second electrodes 106 to maintain optimal signal quality. The one or more alerts may indicate which specific electrode contact location exhibits suboptimal electrical contact, allowing the user to make targeted adjustments to posture or contact pressure at the identified location.

In an exemplary embodiment, the system 102 may block measurement until all electrodes of the pair of first electrodes 104 and the pair of second electrodes 106 have acceptable contact and a stable ECG waveform is detected. The adaptive contact-detection models 112a may continuously evaluate the skin-electrode impedance, the signal-to-noise ratio, and the baseline drift at each defined electrode contact location during an initial contact validation phase. The control unit 112 may prevent data capture and waveform recording until the adaptive contact-detection models 112a confirm that all electrode contact locations meet predetermined contact quality thresholds. The control unit 112 may further verify that a stable waveform is detected before initiating the measurement, where waveform stability may be determined based on consistency of detected cardiac peaks over a predetermined time interval. The measurement blocking validation may reduce the occurrence of repeated electrocardiogram measurements due to suboptimal electrocardiogram data quality, thereby improving efficiency of an electrocardiogram measuring process.

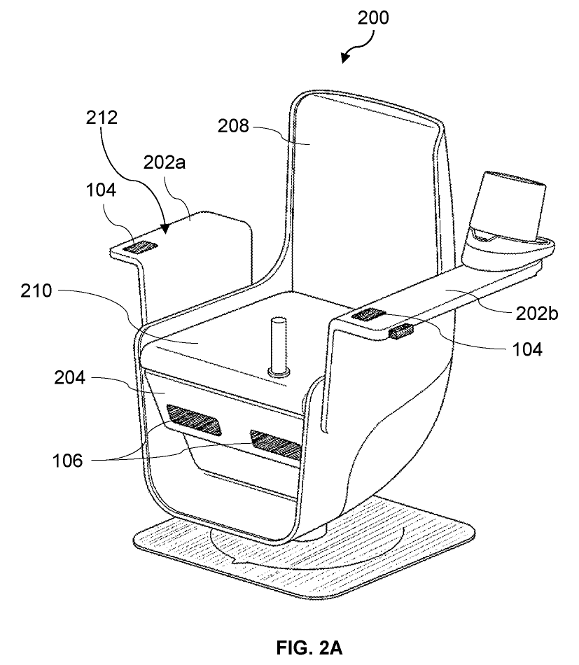

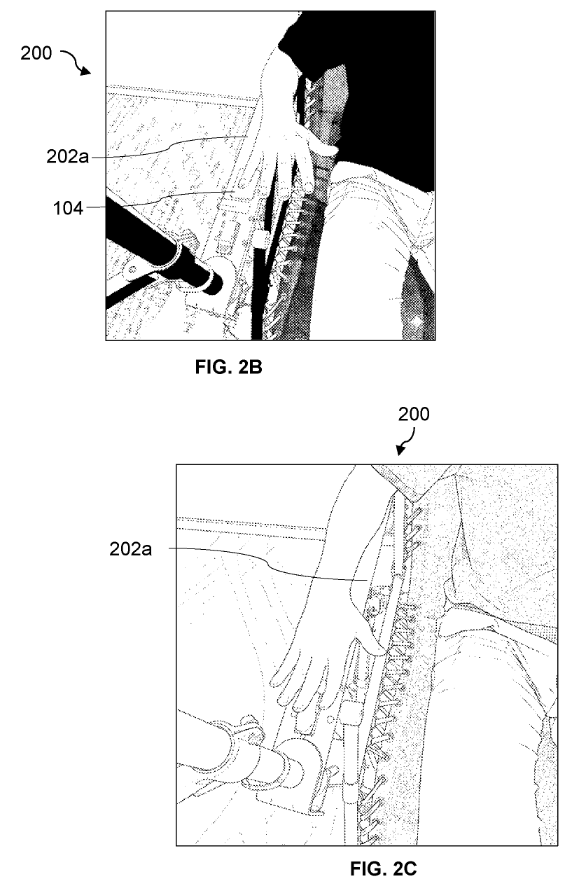

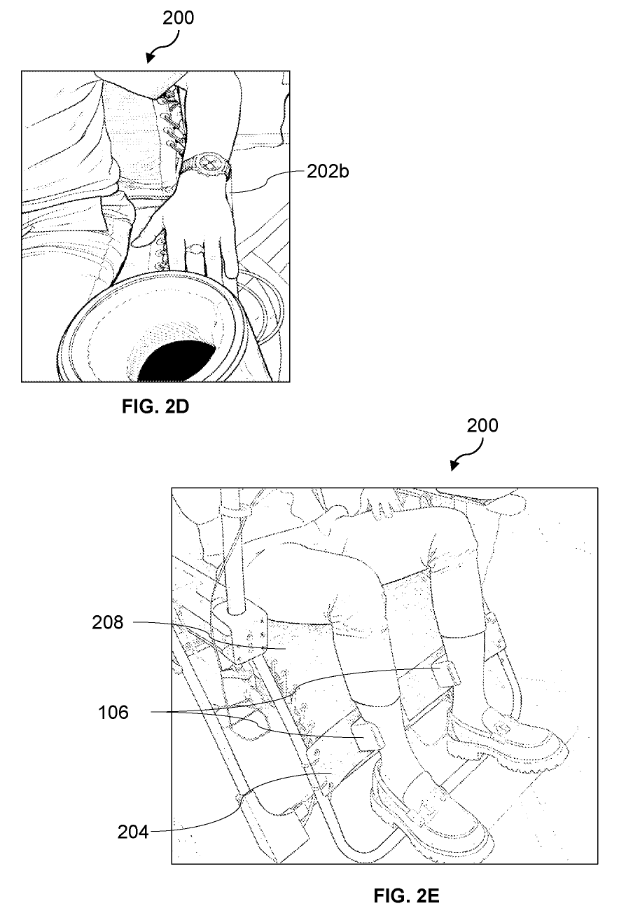

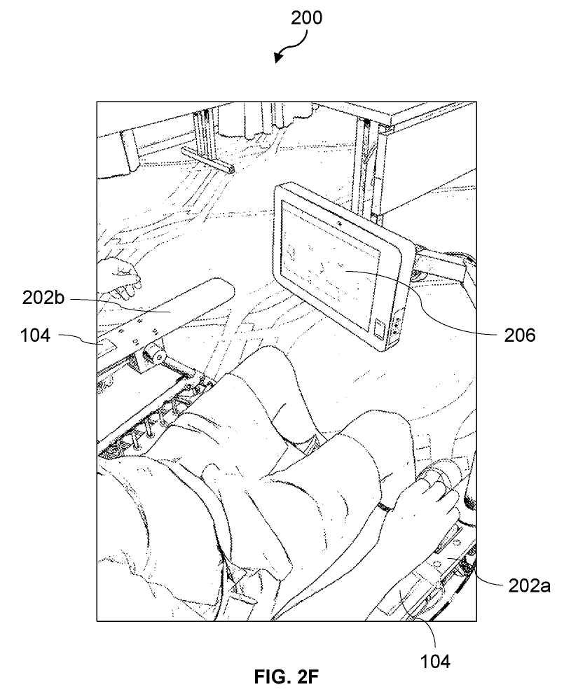

FIG. 2A illustrates an exemplary visual representation 200 of the user support device 208 with the multiple anatomical contact electrodes (104 and 106), in accordance with an embodiment of the present disclosure.

In an illustrative embodiment, the user support device 208 includes opposing armrests (202a and 202b) positioned on opposite sides of one of: a seating structure 210 or a laying structure. A first armrest 202a of the opposing armrests (202a and 202b) may be positioned on one side of the user support device 208, and a second armrest 202b of the opposing armrests (202a and 202b) may be positioned on an opposing side of the user support device 208. The user support device 208 further includes a calf support 204 positioned at a lower portion of one of: the seating structure 210 or the laying structure.

In an illustrative embodiment, the pair of first electrodes 104 may be integrated into the first armrest 202a and the second armrest 202b. Each first electrode of the pair of first electrodes 104 may be positioned on an upper surface 212 of the respective armrest to establish electrical contact with palms or wrists of the user when arms are resting on the opposing armrests (202a and 202b). The pair of second electrodes 106 may be integrated into the calf support 204. The pair of second electrodes 106 may be configured to provide electrical contact with calves or ankles of the user when the user is seated on the user support device 208.

In an illustrative embodiment, the user support device 208 features a backrest portion extending upward from the seating structure 210. The overall configuration of the user support device 208 accommodates a seated user for ECG data acquisition through the pair of first electrodes 104 and the pair of second electrodes 106. The user support device 208 may be implemented as a recliner chair. In an exemplary embodiment, the user support device 208 may comprises, but not limited to, one of: chairs, beds, wheelchairs, recliners, examination tables, and the like. The integration of the pair of first electrodes 104 into the opposing armrests (202a and 202b) and the pair of second electrodes 106 into the calf support 204 may enable ECG monitoring capabilities across various seating and support surface implementations.

FIGS. 2B to 2D illustrate exemplary visual representations 200 depicting opposing armrests (202a and 202b) of the user support device 208 integrated with a pair of first electrodes 104, in accordance with an embodiment of the present disclosure.

FIG. 2E illustrates an exemplary visual representation 200 depicting the calf support 204 along with a pair of second electrodes 106 attached to the user support device 208, in accordance with an embodiment of the present disclosure.

With continued reference to FIGS. 2B and 2C, each first electrode of the pair of first electrodes 104 may comprise an electrically conductive material constructed/ selected from, but not limited to, one of: copper, silver, silver chloride, stainless steel, silver-coated fibers, conductive polymers, and carbon. The pair of first electrodes 104 may be implemented as metal plates. In some cases, the electrodes may be made from biocompatible conductive materials such as medical-grade stainless steel. Medical-grade stainless steel may include SS304L or similar alloys suitable for skin contact applications.

The pair of first electrodes 104 may be positioned on the upper surfaces 212 of the opposing armrests (202a and 202b) at predetermined locations and orientations corresponding to average palm and wrist resting positions of the user. The pair of first electrodes 104 may be configured to establish the electrical contact in response to the user resting arms on the opposing armrests (202a and 202b). The predetermined locations and orientations may be determined based on anthropometric reference data to accommodate a range of user arm lengths and hand sizes.

In an exemplary embodiment, the anthropometric reference data refers to a set of statistically derived dimensional and positional measurements corresponding to anatomical characteristics of a representative user population, including at least one of lower limb length, calf circumference, ankle width, forearm length, wrist position, and seated posture parameters. The anthropometric reference data is utilized to dimension and contour the user support device 208, the opposing armrests (202a and 202b), and the calf support 204 such that the pair of first electrodes 104 and the pair of second electrodes 106 are positioned at predetermined locations and orientations for establishing consistent electrical contact with palms, wrists, calves, or ankles of the user. The application of the anthropometric reference data enables maintenance of stable skin-electrode impedance and reliable acquisition of cardiac biopotential voltage signals during use.

As further shown in FIGS. 2B and 2C, the pair of first electrodes 104 on the opposing armrests (202a and 202b) may have a convex or raised surface profile. The convex or raised surface profile may encourage natural arm placement by the user. The convex or raised surface profile may improve consistency of skin contact between the pair of first electrodes 104 and one of the palms and the wrists of the user without requiring gels, adhesives, or straps.

In some cases, the right leg electrode may be positioned on the second armrest 202b to make contact with a forearm of the user. The right leg electrode may function as a driver electrode for improving noise rejection and signal quality. The right leg electrode may alternatively or additionally be positioned on the calf support 204 to make contact with a right leg calf of the user. In some aspects, the right leg electrode may be configured to establish electrical contact at either the forearm location or the right leg calf location, or at both locations simultaneously, depending on user positioning and system configuration. The driver electrode may provide a reference potential that reduces common-mode interference in the cardiac biopotential voltage signals captured by the measurement electrodes.

In an exemplary embodiment, the pair of first electrodes 104 may be implemented as capacitive electrodes. The capacitive electrodes may detect the cardiac biopotential voltage signals through capacitive coupling without requiring direct galvanic contact with the skin of the user. In some cases, the pair of first electrodes 104 may be implemented as textile-based conductive surfaces integrated into upholstery of the opposing armrests (202a and 202b). The textile-based conductive surfaces may provide a comfortable contact interface while maintaining electrical conductivity for capturing the cardiac biopotential voltage signals.

In some exemplary cases, the pair of first electrodes 104 may be implemented as polymer-coated electrodes. The polymer-coated electrodes may include a conductive polymer layer that provides biocompatibility and corrosion resistance while maintaining electrical conductivity. In some cases, the pair of first electrodes 104 may be implemented as impedance-based sensing elements. The impedance-based sensing elements may monitor skin-electrode impedance characteristics in addition to capturing the cardiac biopotential voltage signals.

In an exemplary embodiment, the calf support 204 along with the pair of second electrodes 106 associated with the system 102. FIG. 2D shows a close-up view of a user's hand positioned on the second armrest 202b of the user support device 208, illustrating natural placement of the user's palm and wrist on the armrest surface. FIG. 2E provides a view of a lower portion of the user support device 208, showing the calf support 204 and the pair of second electrodes 106 positioned to make electrical contact with one of: the calves and the ankles of the user.

With continued reference to FIGS. 2D and 2E, each second electrode of the pair of second electrodes 106 may comprise a semicircular arch configured with a conductive tape. The semicircular arch configuration may extend from the calf support 204 toward lower limbs of the user when the user is seated on the user support device 208. The conductive tape may be constructed from, but not limited to, one of: copper, silver, silver chloride, stainless steel, silver-coated fibers, conductive polymers, carbon, and the like. The conductive tape may facilitate electrical connection with skin of the user at one of the calves and the ankles.

As further shown in FIGS. 2D and 2E, the pair of second electrodes 106 may further comprise a resilient biasing member configured to urge the conductive tape toward a surface of the calf support 204. The resilient biasing member may provide a user-perceptible tactile response upon establishing the electrical contact between the pair of second electrodes 106 and one of the calves and the ankles of the user. The resilient biasing member may be a spring-loaded mechanism that allows controlled movement upon contact. The spring-loaded mechanism may provide tactile feedback to the user, indicating proper contact between the pair of second electrodes 106 and the skin of the user. The spring-loaded mechanism may maintain safe and comfortable pressure across different body types and seating postures of the user.

The calf support 204 may be dimensioned and contoured according to the anthropometric reference data to accommodate placement of one of the calves and the ankles of the user. The dimensioning and contouring of the calf support 204 according to anthropometric reference data may maintain consistent skin-electrode impedance during use. The calf support 204 may allow for comfortable placement of one of the calves and the ankles of the user while ensuring apt electrical contact with the pair of second electrodes 106. The pair of second electrodes 106 may be positioned to naturally align with one of the calves and the ankles of the user when seated on the user support device 208, which may facilitate consistent and reliable electrocardiogram data acquisition.

FIG. 2F illustrates an exemplary visual representation 200 depicting a display unit 206 attached to the user support device 208, in accordance with an embodiment of the present disclosure.

Referring to FIG. 2F, a visual representation 200 depicts a perspective view of a user seated in the user support device 208 during an ECG measurement session. The user support device 208 includes the first armrest 202a and the second armrest 202b positioned on opposite sides of the seating structure. The pair of first electrodes 104 is integrated into the first armrest 202a and the second armrest 202b, with one electrode of the pair of first electrodes 104 visible on each armrest. The user is shown with arms resting on the first armrest 202a and the second armrest 202b, establishing electrical contact between the palms or wrists and the pair of first electrodes 104 for capturing the cardiac biopotential voltage signals.

As further shown in FIG. 2F, the system 102 may further comprise the display unit 206 operatively attached to the user support device 208. The display unit 206 may be positioned anterior to the user. The display unit 206 may be operatively mounted on the first armrest 202a. The positioning of the display unit 206 on the first armrest 202a may allow for positioning of the display unit 206 based on user preference during the ECG measurement session.

With continued reference to FIG. 2F, the display unit 206 may be configured to display the real-time ECG waveform and the one or more alerts. The display unit 206 may present the real-time ECG waveform to confirm successful acquisition of the cardiac biopotential voltage signals. The display unit 206 may also display the one or more alerts generated by the control unit 112 in response to the adaptive contact-detection models 112a identifying suboptimal electrical contact at one or more of the defined electrode contact locations.

The real-time ECG waveform may be transmitted by the control unit 112 to a user interface associated with the one or more edge devices 118. The one or more edge devices 118 may include, but not limited to, one of: a mobile device, a smartphone, a personal digital assistant (PDA), a tablet computer, a phablet computer, a wearable computing device, a virtual reality/augmented reality (VR/AR) device, a laptop, a desktop, and the like. The one or more alerts may also be transmitted to the user interface associated with the one or more edge devices 118. The transmission of the real-time electrocardiogram (ECG) waveform and the one or more alerts to the one or more edge devices 118 may enable the user to monitor the electrocardiogram measurement session from one or more edge devices 118 other than the display unit 206 operatively attached to the user support device 208.

Each electrode of the pair of first electrodes 104 and the pair of second electrodes 106 may be connected to signal acquisition electronics through a detachable connection interface. The pair of first electrodes 104 and the pair of second electrodes 106 may be implemented as modular subassemblies that include an electrode contact element and an associated cable or connector. The detachable connection interface may use pogo pins for establishing electrical connections between the electrode subassemblies and the signal acquisition electronics. In some cases, the detachable connection interface may use spring contacts that provide reliable electrical connections while accommodating manufacturing tolerances and repeated connection cycles. In some cases, the detachable connection interface may use flex interconnects that provide flexible cable-based connections between the electrode subassemblies and the signal acquisition electronics.

The modular subassembly architecture may allow electrode assemblies to be pre-assembled and tested independently before installation into the user support device 208. During final assembly, the electrode modules may be connected to a main electronics unit using plug-in connectors or quick-disconnect interfaces. The modular design may simplify manufacturing by allowing parallel assembly of electrode subassemblies and signal acquisition electronics. The modular design may reduce assembly time by eliminating the need for permanent wiring connections during final assembly. The detachable connection interface may improve serviceability by allowing individual electrodes or cables to be replaced without disassembling the entire user support device 208. The detachable mechanism may support different electrode variants or materials to be used across product configurations without redesigning core electronics of the system 102.

The system 102 may allow for continuous ECG monitoring over an extended period of approximately 40 seconds. The system 102 may record the ECG data for a predefined time duration after stability is achieved. The predefined time duration of approximately 40 seconds may allow capture of multiple cardiac cycles, providing a comprehensive view of heart activity of the user. The control unit 112 may initiate ECG data recording after the adaptive contact-detection models 112a confirm that all electrode contact locations meet predetermined contact quality thresholds and a stable ECG waveform is detected. The system 102 may continuously acquire the cardiac biopotential voltage signals during the predefined

time duration and store the digitized cardiac biopotential voltage signals for subsequent analysis.

The system 102 may process the recorded data to calculate heart rate of the user. The heart rate calculation may be performed by detecting cardiac peaks in the recorded ECG data and determining intervals between successive cardiac peaks. The control unit 112 may compute an average heart rate based on the detected cardiac peak intervals over the predefined time duration.

In an exemplary embodiment, respiratory rate may be derived when the leg electrode is present and connected. The respiration waveform may be derived based on impedance changes measured between the right arm electrode and the left leg electrode. The control unit 112 may inject a low-amplitude, high-frequency excitation signal through the right arm electrode and the left leg electrode and measure resulting impedance variations corresponding to thoracic expansion and contraction during respiration. The impedance changes may modulate the baseline of the cardiac biopotential voltage signals, and the control unit 112 may extract the respiration waveform by applying bandpass filtering to isolate respiratory frequency components from the measured impedance signal. The respiratory rate derivation may be performed by analyzing the extracted respiration waveform to identify respiratory cycles and calculate a respiration rate based on intervals between successive respiratory peaks.

In an exemplary embodiment, during normal operation, the user may establish electrical contact with all four electrodes of the system 102, including the left arm electrode, the right arm electrode, the left leg electrode, and the right leg electrode. When all four electrodes are connected, the system 102 may capture Lead I waveform data, Lead II waveform data, and respiration waveform data. The control unit 112 may compute heart rate based on cardiac peak detection in the Lead I waveform, the Lead II waveform, or both. The control unit 112 may further compute respiration rate based on the respiration waveform derived from impedance measurements between the right arm electrode and the left leg electrode. The normal operating mode may provide comprehensive physiological monitoring including electrocardiogram waveforms, heart rate, and respiration rate.

The system 102 may analyze the ECG data to determine arrhythmia status of the user. The arrhythmia status analysis may include detection of irregular cardiac rhythms, abnormal cardiac peak morphologies, or deviations from normal cardiac timing intervals. The control unit 112 may compare the recorded electrocardiogram data against predetermined criteria for identifying arrhythmia conditions. The arrhythmia status may be displayed to the user on the display unit 206 or transmitted to the one or more edge devices 118 for review by the user or healthcare personnel.

In another exemplary embodiment, the system 102 may use optical cardiac sensing as an alternative implementation. The optical cardiac sensing may detect cardiac activity through photoplethysmography or similar optical measurement techniques. The optical cardiac sensing may provide an alternative or supplementary method for capturing cardiac signals in addition to the electrical contact-based ECG measurement performed by the pair of first electrodes 104 and the pair of second electrodes 106. In some exemplary cases, the optical cardiac sensing may be integrated into the user support device 208 to provide redundant cardiac monitoring capabilities or to enable cardiac signal acquisition when electrical contact with the pair of first electrodes 104 and the pair of second electrodes 106 is not established.

In an exemplary embodiment, the system 102 enables routine and rapid acquisition of the ECG data in office-based clinical settings and primary care environments. The integration of the multiple anatomical contact electrodes (104 and 106) into the user support device 208 facilitates efficient ECG measurement without requiring extensive preparation or electrode attachment procedures. The real-time electrocardiogram data acquired by the system 102 may enable detection of clinically significant cardiac abnormalities, including atrial fibrillation, prior myocardial infarction, and intraventricular conduction abnormalities, in users who may be asymptomatic or not presenting complaint-driven indications. Early identification of such abnormalities may support timely initiation of therapeutic or preventive interventions, including anticoagulation therapy and secondary prevention measures. Furthermore, the ability of the system 102 to perform rapid ECG screening with minimal workflow disruption may facilitate integration of ECG measurements into routine clinical processes while maintaining consistency and reliability of acquired cardiac biopotential voltage signals.

In another exemplary embodiment, the system 102 may operate in a fallback mode when the user is unable to establish electrical contact with the leg electrodes i.e., the pair of second electrodes 106. In the fallback mode, the user may establish electrical contact with the left arm electrode and the right arm electrode while the left leg electrode and the right leg electrode remain unconnected. The adaptive contact-detection models 112a may detect the absence of leg electrode contact and configure the system 102 to operate in the fallback mode. In the fallback mode, the system 102 may capture the Lead I waveform data based on the potential difference between the right arm electrode and the left arm electrode. The control unit 112 may derive heart rate from the Lead I waveform by detecting cardiac peaks and calculating intervals between successive cardiac peaks. The fallback mode may enable continued cardiac monitoring and heart rate computation even when full electrode connectivity is not achievable, thereby improving accessibility for users with mobility limitations or positioning constraints.

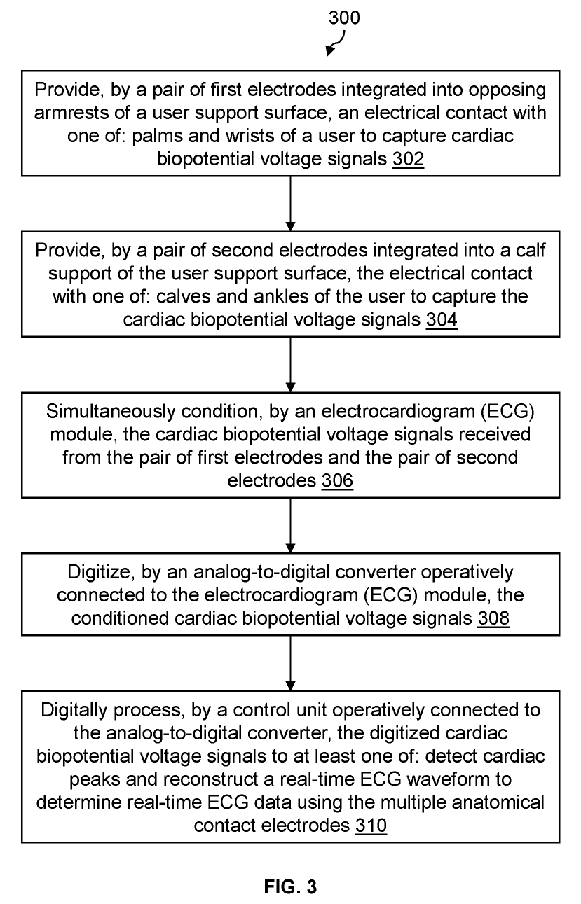

FIG. 3 illustrates an exemplary flowchart of a method 300 for determining the real-time ECG data using the multiple anatomical contact electrodes (104 and 106), in accordance with an embodiment of the present disclosure.

In accordance with another exemplary embodiment of the present disclosure, the method 300 comprises a step 302, a step 304, a step 306, a step 308, and a step 310. The method 300 may be performed using the system 102 described with reference to FIG. 1 and the user support device 208 described with reference to FIGS. 2A through 2F.

With continued reference to FIG. 3, the step 302 comprises providing, by the pair of first electrodes 104 of the multiple anatomical contact electrodes integrated into the opposing armrests (202a and 202b) of the user support device 208, the electrical contact with one of palms and wrists of the user to capture the cardiac biopotential voltage signals. The pair of first electrodes 104 may establish the electrical contact in response to the user resting arms on the opposing armrests (202a and 202b). Each first electrode of the pair of first electrodes 104 may comprise an electrically conductive material constructed from, but not limited to, one of copper, silver, silver chloride, stainless steel, silver-coated fibers, conductive polymers, carbon, and the like. The pair of first electrodes 104 may be positioned on the upper surfaces 212 of the opposing armrests (202a and 202b)

at predetermined locations and orientations corresponding to average palm and wrist resting positions of the user.

As further shown in FIG. 3, the step 304 comprises providing, by the pair of second electrodes 106 of the multiple anatomical contact electrodes integrated into the calf support 204 of the user support device 208, the electrical contact with one of the calves and the ankles of the user to capture the cardiac biopotential voltage signals. Each second electrode of the pair of second electrodes 106 may comprise a semicircular arch configured with a conductive tape constructed from, but not limited to, one of copper, silver, silver chloride, stainless steel, silver-coated fibers, conductive polymers, carbon, and the like. The pair of second electrodes 106 may further comprise a resilient biasing member configured to urge the conductive tape toward the surface of the calf support 204 to provide the user-perceptible tactile response upon establishing the electrical contact. The calf support 204 may be dimensioned and contoured according to anthropometric reference data to accommodate placement of one of the calves and the ankles of the user for maintaining consistent skin-electrode impedance during use.

With continued reference to FIG. 3, the step 306 comprises simultaneously conditioning, by the ECG module 108, the cardiac biopotential voltage signals received from the pair of first electrodes 104 and the pair of second electrodes 106. Simultaneously conditioning the cardiac biopotential voltage signals may comprise differentially amplifying, by the instrumentation amplifier 108a, the cardiac biopotential voltage signals. Simultaneously conditioning the cardiac biopotential voltage signals may further comprise at least one of attenuating baseline drift, power-line interference, and elevated-frequency noise via the at least one analog filter 108b.

The step 308 comprises digitizing, by the analog-to-digital converter 110 operatively connected to the ECG module 108, the conditioned cardiac biopotential voltage signals. The analog-to-digital converter 110 may convert the conditioned cardiac biopotential voltage signals from an analog format to a digital format suitable for processing by the control unit 112.

As further shown in FIG. 3, the step 310 comprises digitally processing, by the control unit 112 operatively connected to the analog-to-digital converter 110, the

digitized cardiac biopotential voltage signals to at least one of detect cardiac peaks and reconstruct the real-time ECG waveform to determine the real-time ECG data using the multiple anatomical contact electrodes (104 and 106).

The method 300 may further comprise monitoring, by the adaptive contact-detection models 112a associated with the control unit 112, at least one of the skin-electrode impedance, the signal-to-noise ratio, and the baseline drift at defined electrode contact locations on the user. The method 300 may further comprise identifying, by the adaptive contact-detection models 112a, suboptimal electrical contact based on the monitoring. The method 300 may further comprise generating, by the control unit 112, the corrective output as one or more alerts in response to identifying the suboptimal electrical contact.

The method 300 may further comprise transmitting, by the control unit 112, the one or more alerts prompting the user to adjust one of the posture and the contact of at least one of the palms, the wrists, the calves, and the ankles with the pair of first electrodes 104 and the pair of second electrodes 106 to maintain optimal signal quality. The one or more alerts may comprise at least one of one or more visual alerts and one or more audio alerts transmitted in real-time.

The method 300 may further comprise displaying, by the display unit 206 operatively attached to the user support device 208, the real-time ECG waveform and the one or more alerts. The method 300 may further comprise transmitting, by the control unit 112, the real-time ECG data to the cloud server 114 for at least one of storage, processing, analysis, and medical records.

Numerous advantages of the present disclosure may be apparent from the discussion above. In accordance with the present disclosure, the system for determining real-time ECG data using the multiple anatomical contact electrodes. The system enables convenient, non-intrusive ECG monitoring in the seated position, making it easy for the one or more users to conduct regular heart assessments without medical assistance. The multi-contact electrodes provide the reliable, continuous ECG data. Overall, the configuration of the system promotes accessible health monitoring, particularly beneficial for the one or more users in non-clinical settings.

The system utilizes multiple anatomical contact electrodes positioned at upper-limb and lower-limb locations, enabling simultaneous acquisition of cardiac biopotential voltage signals from spatially distributed body regions. This configuration improves signal redundancy, enhances vector-based signal interpretation, and increases robustness against localized signal degradation compared to single-site or limited-contact systems.

Integration of electrodes into ergonomically dimensioned armrests and calf supports, in combination with resilient biasing members, maintains consistent contact pressure and stable the skin-electrode impedance. This reduces signal fluctuations caused by posture changes, muscle movement, and involuntary user motion, thereby improving waveform consistency over extended monitoring durations.

The incorporation of the one or more adaptive contact-detection models enables continuous monitoring of impedance, the signal-to-noise ratio, and the baseline drift at defined electrode locations. Automated identification of suboptimal contact conditions and generation of corrective alerts minimizes signal degradation and reduces the need for repeated measurement sessions, which is not addressed in conventional seating-based ECG systems.

The ECG module employs differential amplification and multi-stage analog filtering to suppress baseline wander, power-line interference, and high-frequency noise prior to digitization. This improves signal integrity at the hardware level, reducing dependence on computationally intensive post-processing techniques used in prior art systems.

The use of a dedicated analog-to-digital converter with defined sampling frequency and resolution preserves temporal and amplitude characteristics of cardiac signals. Accurate digitization enables reliable detection of cardiac peaks and waveform reconstruction, thereby improving diagnostic reliability compared to portable or low-resolution ECG devices.

Real-time generation of one or more alerts based on detected contact degradation establishes a closed-loop interaction between the user and the system. This feedback mechanism enables immediate corrective action, maintaining optimal

measurement conditions without clinical supervision, which is not provided in traditional ECG furniture systems.

Unlike conventional adhesive-based ECG systems, the present system eliminates the need for electrode attachment, conductive gels, and skin preparation. The ECG data acquisition is initiated through normal seated posture, thereby reducing setup time and improving usability in non-clinical environments.

The integrated seating configuration enables repeated or continuous ECG measurements in relaxed environments such as homes and offices. Stable electrode positioning and automated quality control permit long-duration monitoring without frequent recalibration, addressing limitations of short-session diagnostic systems.

The modular architecture comprising the ECG module, analog-to-digital converter, control unit, display unit, and cloud interface enables scalable deployment and integration with remote healthcare platforms. This facilitates longitudinal data analysis, remote diagnostics, and medical record management, extending beyond the capabilities of conventional standalone ECG devices.

Coordinated placement of upper-limb and lower-limb electrodes combined with adaptive signal analysis reduces sensitivity to motion-induced artifacts. This improves measurement reliability in real-world usage scenarios where complete immobility cannot be guaranteed.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are

not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words "comprising," "having," "containing," and "including," and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

What is claimed is:

1. A system for determining real-time electrocardiogram (ECG) data using multiple anatomical contact electrodes, the system comprising:
a pair of first electrodes of the multiple anatomical contact electrodes integrated into opposing armrests of a user support device, configured to provide an electrical contact with one of: palms and wrists of a user for capturing cardiac biopotential voltage signals;
a pair of second electrodes of the multiple anatomical contact electrodes integrated into a calf support of the user support device, configured to provide the electrical contact with one of: calves and ankles of the user for capturing the cardiac biopotential voltage signals;
an electrocardiogram (ECG) module electrically coupled to the pair of first electrodes and the pair of second electrodes, configured to simultaneously condition the cardiac biopotential voltage signals received from the pair of first electrodes and the pair of second electrodes;
an analog-to-digital converter operatively connected to the electrocardiogram (ECG) module, configured to digitize the conditioned cardiac biopotential voltage signals; and
a control unit operatively connected to the analog-to-digital converter, configured to digitally process the digitized cardiac biopotential voltage signals to at least one of: detect cardiac peaks and reconstruct a real-time electrocardiogram (ECG) waveform for determining real-time electrocardiogram (ECG) data using the multiple anatomical contact electrodes.

2. The system of claim 1, wherein each first electrode of the pair of first electrodes comprises an electrically conductive material constructed from one of: copper, silver, silver chloride, stainless steel, silver-coated fibers, conductive polymers, and carbon.

3. The system of claim 1, wherein each second electrode of the pair of second electrodes comprises a semicircular arch configured with a conductive tape constructed from one of: the copper, the silver, the silver chloride, the stainless steel, the silver-coated fibers, the conductive polymers, and the carbon,
the pair of second electrodes further comprises a resilient biasing member configured to urge the conductive tape toward a surface of the calf support to provide a user-perceptible tactile response upon establishing the electrical contact.

4. The system of claim 1, wherein the pair of first electrodes is positioned on upper surfaces of the opposing armrests at predetermined locations and orientations corresponding to average palm and wrist resting positions of the user, the pair of first electrodes configured to establish the electrical contact in response to the user resting arms on the opposing armrests.

5. The system of claim 1, wherein the calf support is dimensioned and contoured according to anthropometric reference data to accommodate placement of one of: the calves and the ankles of the user for maintaining consistent skin-electrode impedance during use.

6. The system of claim 1, wherein the electrocardiogram (ECG) module comprises:
an instrumentation amplifier configured to differentially amplify the cardiac biopotential voltage signals; and
at least one analog filter configured to at least one of: attenuate baseline drift, power-line interference, and elevated-frequency noise.

7. The system of claim 1, wherein the control unit includes one or more adaptive contact-detection models configured to:
monitor at least one of: the skin-electrode impedance, signal-to-noise ratio, and the baseline drift at defined electrode contact locations on the user;
identify suboptimal electrical contact based on the monitoring; and
generate a corrective output as one or more alerts in response thereto.

8. The system of claim 1, wherein the control unit is configured to transmit the one or more alerts prompting the user to adjust one of: a posture and a contact of at least one of: the palms, the wrists, the calves, and the ankles with the pair of first electrodes and the pair of second electrodes to maintain optimal signal quality,
the one or more alerts comprise at least one of: one or more visual alerts and one or more audio alerts transmitted in real-time.

9. The system of claim 1, further comprising a display unit operatively attached to the user support device, configured to display the real-time electrocardiogram (ECG) waveform and the one or more alerts.

10. The system of claim 1, wherein the control unit is configured to transmit the real-time electrocardiogram (ECG) data to a cloud server for at least one of: storage, processing, analysis, and medical records.

11. A method for determining real-time electrocardiogram (ECG) data using multiple anatomical contact electrodes, the method comprising:
providing, by a pair of first electrodes of the multiple anatomical contact electrodes integrated into opposing armrests of a user support device, an electrical contact with one of: palms and wrists of a user to capture cardiac biopotential voltage signals;
providing, by a pair of second electrodes of the multiple anatomical contact electrodes integrated into a calf support of the user support device, the electrical contact with one of: calves and ankles of the user to capture the cardiac biopotential voltage signals;
simultaneously conditioning, by an electrocardiogram (ECG) module, the cardiac biopotential voltage signals received from the pair of first electrodes and the pair of second electrodes;
digitizing, by an analog-to-digital converter operatively connected to the electrocardiogram (ECG) module, the conditioned cardiac biopotential voltage signals; and
digitally processing, by a control unit operatively connected to the analog-to-digital converter, the digitized cardiac biopotential voltage signals to at least one of: detect cardiac peaks and reconstruct a real-time electrocardiogram (ECG) waveform to determine real-time electrocardiogram (ECG) data using the multiple anatomical contact electrodes.

12. The method of claim 11, wherein each first electrode of the pair of first electrodes comprises an electrically conductive material constructed from one of: copper, silver, silver chloride, stainless steel, silver-coated fibers, conductive polymers, and carbon.

13. The method of claim 11, wherein each second electrode of the pair of second electrodes comprises a semicircular arch configured with a conductive tape constructed from one of: the copper, the silver, the silver chloride, the stainless steel, the silver-coated fibers, the conductive polymers and the carbon,
the pair of second electrodes further comprises a resilient biasing member configured to urge the conductive tape toward a surface of the calf support to provide a user-perceptible tactile response upon establishing the electrical contact.

14. The method of claim 11, wherein
positioning the pair of first electrodes on upper surfaces of the opposing armrests at predetermined locations and orientations corresponding to average palm and wrist resting positions of the user, the pair of first electrodes establishing the electrical contact in response to the user resting arms on the opposing armrests.

15. The method of claim 11, wherein the calf support is dimensioned and contoured according to anthropometric reference data to accommodate placement of one of: the calves and the ankles of the user for maintaining consistent skin-electrode impedance during use.

16. The method of claim 11, wherein simultaneously conditioning the cardiac biopotential voltage signals comprises:
differentially amplifying, by an instrumentation amplifier, the cardiac biopotential voltage signals; and
at least one of: attenuating baseline drift, power-line interference, and elevated-frequency noise via at least one analog filter.

17. The method of claim 11, further comprising:
monitoring, by one or more adaptive contact-detection models associated with the control unit, at least one of: the skin-electrode impedance, signal-to-noise ratio, and the baseline drift at defined electrode contact locations on the user;
identifying, by the one or more adaptive contact-detection models, suboptimal electrical contact based on the monitoring; and
generating, by the control unit, a corrective output as one or more alerts in response to identifying the suboptimal electrical contact.

18. The method of claim 11, further comprising:
transmitting, by the control unit, the one or more alerts prompting the user to adjust one of: a posture and a contact of at least one of: the palms, the wrists, the calves, and the ankles with the pair of first electrodes and the pair of second electrodes to maintain optimal signal quality,
wherein the one or more alerts comprise at least one of: one or more visual alerts and one or more audio alerts transmitted in real-time.

19. The method of claim 11, further comprising:
displaying, by a display unit operatively attached to the user support device, the real-time electrocardiogram (ECG) waveform and the one or more alerts.

20. The method of claim 11, further comprising:
transmitting, by the control unit, the real-time electrocardiogram (ECG) data to a cloud server for at least one of: storage, processing, analysis, and medical records.
